(19) United States
(12) Patent Application Publication
(10) Pub. No.: US 2018/0256842 A1
(43) Pub. Date: Sep. 13, 2018

(54) HOUSING OF A HEAT AND MOISTURE EXCHANGER AND METHOD FOR DETACHING A COVER FROM A HOUSING

(71) Applicant: Andreas Fahl Medizintechnik - Vertrieb GmbH, Cologne (DE)

(72) Inventor: Andreas Fahl, Köln (DE)

(21) Appl. No.: 15/537,512

(22) PCT Filed: Dec. 18, 2015
(86) PCT No.: PCT/EP2015/080634

§ 371 Date: Mar. 22, 2018

(30) Foreign Application Priority Data
Dec. 18, 2014 (DE) 10 2014 018 677.6

(51) Int. Cl. A61M 16/04; A61M 16/10

(52) U.S. Cl. CPC A61M 16/0468; A61M 16/1045; A61M 16/047

(57) ABSTRACT

The invention relates to a housing (10) of a heat and moisture exchanger for tracheotomized and laryngectomized patients, comprising a cylindrical housing casing (12) and a filter retaining device, said filter retaining device (14) having at least one connecting piece (16) which is associated with a base surface of the housing (10) and the cylindrical housing casing (12) having at least three notched portions (18) that allow the cylindrical housing casing (12) to be dented in a defined fashion.

The invention relates to a housing of a heat and moisture exchanger for tracheotomy and laryngectomy patients, comprising a cylinder housing casing and a filter retaining device, as well as a method for releasing a cover from such a housing.

Heat and moisture exchangers for tracheotomy patients are known in general from the prior art. These heat and moisten the respiratory air by means of a filter usually disposed in a cassette or housing. The filter stores the heat and moisture of the exhaled air and feeds it back into the respiratory air during inhalation.

A disadvantage with the known heat and moisture exchanger housings is that it is difficult or complicated to remove potential build-up. Thus, the PROVOX HME CAP from Atos Medical, for example, is known, which can be fitted onto a heat and moisture exchanger. These are intended to enable a closing of the filter, or tracheostoma, with the finger, such that it is possible to speak via a voice prosthesis.

The aforementioned cap comprises a curved titanium ring having a rigid construction and a breathing hole. A disadvantage with the known combination composed of a filter or filter cassette and a cap is that the heat and moisture exchanger filter is disposed directly beneath the cap. On one hand, this has a disadvantageous appearance, and on the other hand, when closing the breathing hole in order to initiate the speech process, the finger of the user comes in direct contact with the filter, which can lead to contamination. A further disadvantage with the known heat and moisture exchanger is that it is very difficult to remove build-up from the housing, such that elderly patients may have difficulties using it.

The object of the invention is thus to overcome known disadvantages from the prior art, and to create an improved housing for a heat and moisture exchanger for tracheotomy and laryngectomy patients.

The object is achieved according to the invention by means of a housing according to Claim 1 and a method for releasing a cover from a housing according to Claim 13. Further advantageous designs can be derived from the following description, the drawings, and the dependent Claims. The individual features of the different designs are not limited thereto, however, but rather, can be combined with one another and with other features to obtain other designs.

A housing for a heat and moisture exchanger for tracheotomy and laryngectomy patients comprising a cylindrical housing casing is proposed. The housing also has a filter retaining device, wherein the filter retaining device comprises at least one web, allocated to a base surface of the housing. The cylindrical housing casing has at least three notches, which are designed in particular as integral hinges, which enable a denting of the cylindrical housing casing in a defined manner.

Advantageously, the housing can be placed in or on a tracheostoma bandage, a tracheostoma button and/or a tracheostoma cannula. The housing is preferably cylindrical, wherein a base surface of the housing is formed by the filter retaining device. The filter retaining device has one or more webs, in particular such that the webs are interconnected.

The cylindrical housing casing furthermore has at least three notches, which are integral hinges in a preferred design. It is provided in another design that the notches are designed as defined breaking points. It is also preferred that the notches represent weakened points in the material. The notches are preferably distributed over the cylindrical housing casing such that when pressure is applied, in particular finger pressure, to the cylindrical housing casing, it becomes dented. Denting, as set forth in the invention, means that the sections of the cylindrical housing casing defined by the notches are moved toward one another, and in particular, are pressed radially inward. It is also advantageous that the notches in one embodiment extend over the cylindrical housing casing, at least over a partial height of the housing, from the distal toward the proximal. More preferably, the notches extend over the entire height from the distal to the proximal, wherein the notches pass through one or both cylindrical housing end surfaces on at least one side, or, respectively, the notches extend to the proximal and/or distal inner edges of the cylindrical housing end surfaces. In a preferred design, at least one, preferably all, of the notches extend on an inner surface of the cylindrical housing casing. Furthermore, one embodiment provides that at least one notch is provided on an outer surface of the cylindrical housing casing. The advantage with notches on the inner surface of the cylindrical housing casing is that these are not visible when the heat and moisture exchanger is in use, such that the heat and moisture exchanger exhibits a uniform appearance. It is furthermore advantageous that the notches on the inner surface, instead of notches formed on the outer surface, enable a precise denting to be obtained.

The advantage with the housing describe above is that if a cover or other component is disposed on the housing by means of a snap-on connection, it can be easily released from the snap-on connection, when a pressure, in particular a finger pressure, is exerted radially on the cylindrical housing casing. The cylindrical housing casing then becomes dented in a defined manner, such that it is released from a snap-on connection with another component, in particular a cover.

It is provided in a preferred design that the notches are disposed at an angle of approx. 15° to 75°, more preferably approx. 50° to 70°, more preferably approx. 60°, to one another. The arrangement of the notches, and in particular the number of notches, determine the extent of the denting, and the resistance exerted by the cylindrical housing casing when pressure is applied thereto.

If the term “approx.” is used in the framework of the invention, this is to be understood to comprise a tolerance range regarded as normal in this field by a person skilled in the art, in particular a tolerance range of ±20%, preferably ±10% is provided. The term “substantially” also implies a tolerance range that is acceptable to a person skilled in the art taking economic and technical aspects into consideration, such that the feature in question can still be recognized as such.

It is provided in another design that the notches on the cylindrical housing casing are distributed over an angle of approx. 90° to approx. 180°, preferably approx. 100° to approx. 160°, more preferably approx. 120°.

It is provided in a particularly preferred design that the notches are disposed on the inner surface of the cylindrical housing casing. It is provided in another design that at least one notch is disposed on the outer surface of the cylindrical housing casing. The notches are reductions in the thickness of the material, or a tapering of the cross section of the cylindrical housing casing, disposed on one or both sides of the cylindrical housing casing. Different properties, in particular the denting, can be obtained through the different designs of the notches, e.g. having a triangular cross section or a smooth transition or uniform transition to the material thickness of the rest of the cylindrical housing casing. Tensile and compression forces are exerted on the outer and inner surfaces of the cylindrical housing casing through the denting, or the exertion of radial forces on the cylindrical casing. These tensile and compression forces can be converted to deformations in a defined or targeted manner by the material thinning or notches. In particular, it is provided that the denting takes place more easily, and to a greater extent, than with the same pressure exerted on a cylindrical housing casing without such notches. In one design, in which the notches are designed as defined breaking points, the defined breaking thereof should be achieved, in particular, through light pressure to the cylindrical housing casing. In particular, the notches are designed such that injuries are prevented that may be caused by sharp edges and/or uncontrolled breaking, e.g. through the application of too much force, which would make it difficult to control the manipulation thereof.

It is provided in another variation of the housing that the filter retaining device comprises a stop for the denting of the cylindrical housing casing. The stop is formed in particular such that it is disposed opposite the expected greatest denting, i.e. the greatest material displacement. The notches are preferably disposed on the cylindrical housing casing such that a denting, i.e. a material displacement, of at least a portion of the cylindrical housing casing is directed toward the stop. It is provided in another design that the notches face the stop radially. In one design, the stop is designed as a web disposed substantially concentric to the cylindrical housing casing, in particular in the form of a circular ring segment. The stop is preferably designed such that the cylindrical housing casing can only be pressed inward so far that either the cylindrical housing casing does not break, or if a break is intended, it is limited to the region of the notches, in order to prevent a complete breaking, for example.

In the framework of the invention, a defined breaking point, or a break, is understood to be a breaking point, also referred to as a white break point in a plastic component. White breaks are small regions, in which the boundary surfaces are bridged by individual, extremely extended, material strands. These extremely extended material strands are called “crazes,” and comprise an initial damage to the material.

The filter device can be a part of a web, or preferably disposed on the web. In particular, the stop is a protrusion disposed on the web, or is formed by at least one web.

The housing is preferably made of a single material. In another design, the housing comprises numerous materials. It is also preferred that the housing has a single-piece construction. It is provided in another design that the housing comprises numerous pieces. It is particularly preferred that the housing is made of a material selected from a group comprising at least polypropylene and/or polyvinyl chloride. It is provided in one design that the housing is produced by means of a generative production method, e.g. by means of a 3D printer.

It is provided according to another embodiment that the filter retaining device has a number of webs. The number of webs can be disposed parallel, orthogonal, or in a geometric form, to one another. It is provided in particular that the webs are disposed radially. The webs have web ends, which are connected to the cylindrical housing casing and/or other webs according to one design. It is provided according to a particularly preferred design that web ends of the webs are distributed on an inner circumference of the cylindrical housing casing over an angle of approx. 120° to approx. 200°. It is provided in particular that web ends of the webs are distributed on an inner circumference of the cylindrical housing casing over an angle of approx. 180°. This means implicitly that the rest of the cylindrical casing is free of webs, and particularly preferably provides space for the notches for the denting. Thus, it is provided according to one design that the housing has a number of webs, wherein the web ends are distributed on the inner circumference of the cylindrical housing casing over an angle of approx. 180°, and at least three notches are disposed in the cylindrical housing casing on the other half of the cylindrical casing, which are disposed, either likewise over an angle of approx. 180°, or preferably within an angle of approx. 120°, over the remaining part of the cylindrical housing casing, i.e. the partial circumference of the cylindrical housing casing having no webs.

It is provided in a particularly preferred design that the housing has a cover receiver for a cover. The cover is a protection, in particular, for a filter, which is intended on one hand to prevent the filter from falling out, and on the other hand is intended to prevent direct visual contact and/or direct physical contact with the filter. The cover is preferably provided with recesses. The cover receiver is preferably designed as an at least partially circumferential beading, disposed on the side of the cylindrical housing casing disposed opposite the filter retaining device. The cover receiver, in particular the beading, can have interruptions, or it can be distributed only partially on the circumference of the cylindrical housing casing. The cover receiver is advantageously interrupted by the notches. It is provided in another design that the cover receiver is disposed on an inner and/or outer circumference of the cylindrical housing casing. Particularly preferably, the cover receiver is a snap-on device disposed on the outer surface of the cylindrical housing casing. Because the cover receiver is disposed on the outer surface of the cylindrical housing casing, the snap-on device of the cover receiver can be guided out of a snap-in groove of the cover when the cylindrical housing casing is dented by means of a radial pressure applied thereto, and the cover is thus released from the housing.

As a matter of course, it is provided in one design that the cover is snapped onto the housing. In particular, a method for releasing a cover from a housing, as described above, is proposed, wherein a force is applied, perpendicular to an outer surface of a cylindrical housing casing, in particular radially thereto, such that the cylindrical housing casing is dented in a defined manner, due to the notches, and the cover is released from the snap-on connection.

Furthermore, a cover for a housing of a heat and moisture exchanger for tracheotomy and laryngectomy patients is proposed, which comprises a cover rim and an at least partially circular, circumferential first snap-on element on the inside of the cover rim, and a number of holes passing through cover. The corresponding housing preferably has a likewise circular snap-on element, or a circular cover receiver, which interacts with the first snap-on element of the cover. This has the advantage that the cover is rotatably disposed on the housing.

The cover preferably comprises a cover surface, or a cover plate, which forms, according to one design, a substantially circular disk. Furthermore, the cover has a cover rim, which particularly preferably extends away from the cover plate. In this manner, a cylindrical casing is formed below the plate, i.e. a cover rim extending toward the housing when the cover is in use. The cover rim preferably also comprises the radial surface of the cover plate.

The first snap-on element is designed according to one design as a beading or projection. The first snap-on element can be designed thereby as a continuous circular beading on the inside of the cover rim. It is provided in another design that the first snap-on element has a number of beadings or projections, which are disposed on the inside of the cover rim.

The cover has a number of holes passing through the cover, which enable the user to inhale and exhale sufficient air, and in particular to conduct said air through a filter disposed beneath the cover. The cover, in particular the cover plate, advantageously has two or more holes. It is particularly preferably provided that approx. 30% to approx. 60% of the cover surface is penetrated by holes.

One design is particularly preferably provided in which holes pass through a cover surface of the cover at an angle of approx. 30° to approx. 60°, preferably approx. 45°, to the perpendicular. The cover surface is, in particular, the flat surface of the cover, the perpendicular to which is substantially oriented distally when being worn. The holes or perforations thus run at an angle, or diagonally, through the cover. This has the advantage that a direct passage to, or visibility of, the filter or other devices lying beneath the cover can be prevented. Moreover, it is possible for the user to breath well and safely through the cover. Another advantage with the embodiment described above is that a design that is attractive to the user can be obtained. Another advantage of the angled or diagonal holes or bores through the cover is that, in particular when people approach the user, a blowing on third parties is avoided. The air can be advantageously deflected downwards or to the side through this design.

If directions are used in the framework of the description of the invention, these are to be understood with respect to the normal use of the heat and moisture exchanger. Directional terminology is used in the manner normally used in anatomy. The term “distal” is understood, as set forth in the present invention, with respect to a feature of the device according to the invention, to be an arrangement or use thereof at a distance to, or facing away from or lying opposite a tracheostoma bandage or a tracheal cannula, in general a skin surface of a person who, in particular, wears such an attachment means for the device according to the invention. Distal is preferably understood to mean a direction away from the body of the patient. The term “proximal” is understood, as set forth in the present invention, with respect to a feature of the device according to the invention, to be an arrangement or use thereof close to, or facing, or adjacent to a tracheostoma bandage or a tracheal cannula, in general a skin surface of a person who, in particular, wears such an attachment means for the device according to the invention. Proximal is preferably understood to be a direction toward the body of the patient. Transverse is preferably a lateral direction, running toward the right and/or left of a median plane, or a median line. Longitudinal is preferably understood to be a direction along the median plane or median line, in particular the longitudinal axis of the body, running upward or downward. Superior is preferably understood to mean a relative direction along or parallel to the longitudinal axis of the body, above a reference point. Inferior is preferably understood to mean a relative direction along or parallel to the longitudinal axis of the body, below a reference point.

It is provided in another embodiment that at least a portion of the holes are bores. As set forth in the invention, bores are understood to be round, circular or oval holes, which pass entirely through the cover, or penetrate it. The bores can pass through the cover orthogonally to the cover surface, or at an angle of approx. 30° to approx. 60°, preferably approx. 45°, to a perpendicular to the cover surface. It is provided in another design that the holes are disposed in the manner of a grid on the cover surface. In particular, bores or otherwise formed holes are provided. Another design provides that the holes have a cross section that changes over its length, or a changing clearance. In particular, the holes become wider in the distal or proximal direction. It is likewise particularly preferred that at least a portion of the holes are slits. It is provided in another design that all of the holes are designed as slits. It is furthermore advantageous that the slits, as mentioned above, are diagonal or angled, and/or have cross sections that change, in particular over the thickness of the cover, from the distal to the proximal.

It is provided according to another design, that lamellas are disposed between the holes or slits. The lamellas can be formed in another design through the provision of the slits.

It is provided in another design of the cover that the cover rim comprises at least a partially circumferential second snap-on element on the outside. The second snap-on element is designed, in particular, as a completely or partially encompassing beading or other type of protrusion. Furthermore, numerous protrusions are advantageously provided, which collectively form the snap-on element. The second snap-on element on the outside of the cover rim is provided in particular for receiving a shield, in particular a shield that simplifies a closing of the cover, or the breathing passage.

Moreover, a housing to be attached to the cover described above is proposed, wherein the housing has a snap-on receiver for receiving a first snap-on element of the cover.

It is advantageously proposed that the cover can be attached to the housing by means of the first snap-on connection described above, composed in particular of the snap-on element of the cover and the snap-on receiver of the housing, or at least comprises these individual components. As stated above, it is advantageously provided that the snap-on connection is designed such that the cover can rotate on the housing.

Moreover, a shield to be attached to the cover is proposed, wherein the shield comprises an at least partially encompassing inner snap-on receiver for a second snap-on element of the cover. The cover can preferably be attached to the shield by means of the second snap-on connection, composed of the second snap-on element of the cover and the snap-on receiver of the shield. The shield is preferably designed such that it has a breathing hole, which is substantially as large as the diameter of the cover. It is provided in another design that the shield is approx. 1% to approx. 5% smaller than the diameter of the cover. It is particularly preferably provided that the breathing hole can be disposed centrally over the cover. It is also provided in one design that the shield has a recess, or a funnel-shaped design, surrounding the breathing hole. The funnel-shaped design can be either circular or oval, in order to facilitate, in particular, the application of a finger. The funnel-shaped design is advantageous, because it guides the finger to the breathing hole when it is intended that the breathing hole be closed. The user lays the finger on the breathing hole in order to guide the exhaled air through a voice prosthesis. As a result of the proposed design, a lighter sealing is necessary in order to obtain a sealing closure, in particular through light finger pressure applied to the shield, or the heat and moisture exchanger.

It is provided in a particularly preferred design that the shield and/or the cover are made of a rubbery-elastic plastic or an elastic material. By way of example, it is provided that the shield and/or the cover comprise a material selected from a group comprising at least one metal, polypropylene and/or polyvinyl chloride. It is provided in one design that the shield and/or the cover are produced by means of a generative production method, e.g. by means of a 3D printer.

A further aspect of the proposed heat and moisture exchanger is an assembly kit comprising at least a number of housings, as described above, a number of covers, as described above, and a number of shields, as described above. The assembly kit for a heat and moisture exchanger preferably contains different housings and/or different covers and/or different shields. The advantage with the proposed assembly kit is that the medical supplying, or the supplying of a heat and moisture exchanger, can be adapted to the individual user. The assembly kit is distinguished in particular in that it is provided in one design that the housing is disposable. This has the advantage that instead of having to replace or dispose of the entire heat and moisture exchanger, individual components can be reused after cleaning, and only the housing, or just the filter in another design, which is dedicated to the housing, is replaced. It is provided in another design that all of the components of the assembly kit can be reused. It is provided in another variation that the shield can come in a number of different colors or shapes. By way of example, the shapes seen from above can be substantially round or substantially oval. Different materials or combinations of materials for the shield are also provided in further designs. Thus, different elasticities are provided, for example, for different purposes, or needs of the user. It is provided in another embodiment that the shield have different breathing holes and/or funnel-shapes.

The assembly kit in one design is designed such that the cover has a number of differently shaped holes. Thus, holes are provided having a round or oval cross section. Other designs provide that a further number of holes pass straight through the cover, and/or pass through the cover diagonally or at an angle.

It is provided in another design that the cover has different colors. The assembly kit has the advantage that not only the medical performance can be tailored to the user, but also the design can be configured to the tastes of the user.

Further advantageous designs can be derived from the following drawings. The illustrations depicted therein are not, however, to be interpreted as limiting, but rather, the features described therein can be combined with one another and with the features described above, to form further designs. Moreover, it should be noted that the reference symbols given in the description of the figures do not limit the scope of protection for the present invention, but refer only to the exemplary embodiments shown in the figures. Identical components or components having the same function have the same reference symbols in the following.

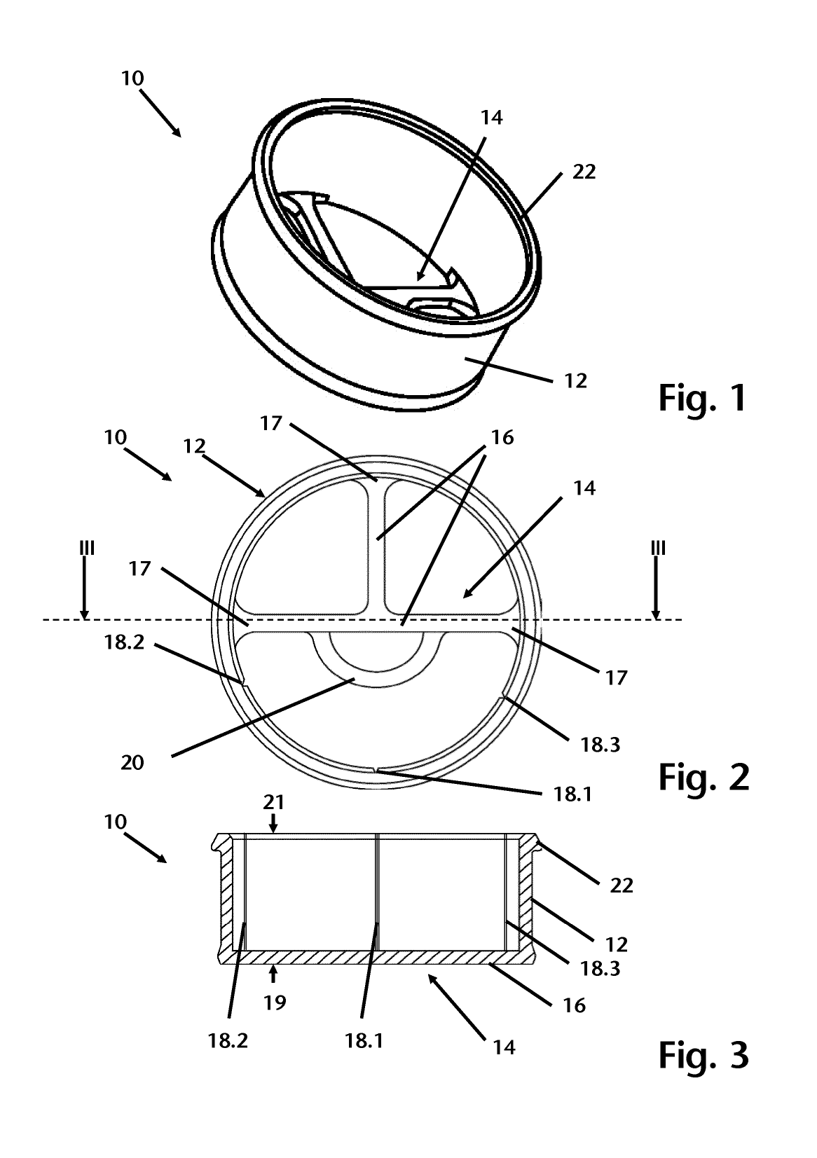

FIG. 1 shows a housing;

FIG. 2 shows the housing seen from above;

FIG. 3 shows the housing from FIG. 2 in a sectional view;

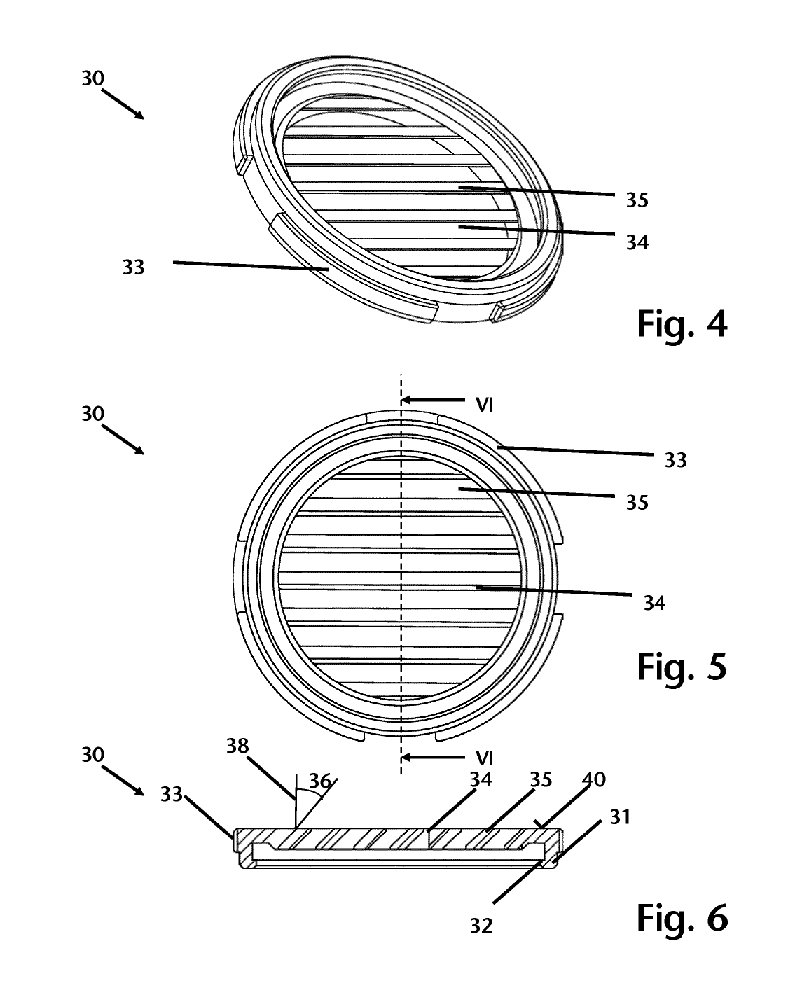

FIG. 4 shows a cover with lamellas;

FIG. 5 shows a cover seen from above;

FIG. 6 shows a sectional view of the cover in FIG. 5;

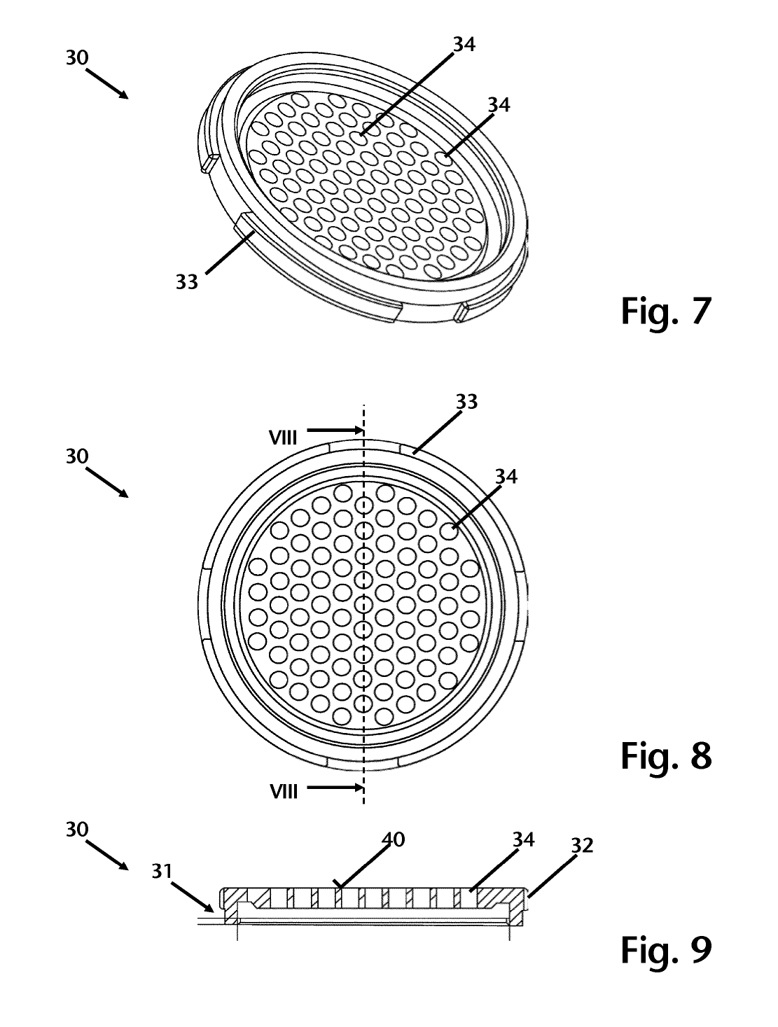

FIG. 7 shows another version of a cover, having bores;

FIG. 8 shows the cover from FIG. 7 seen from above;

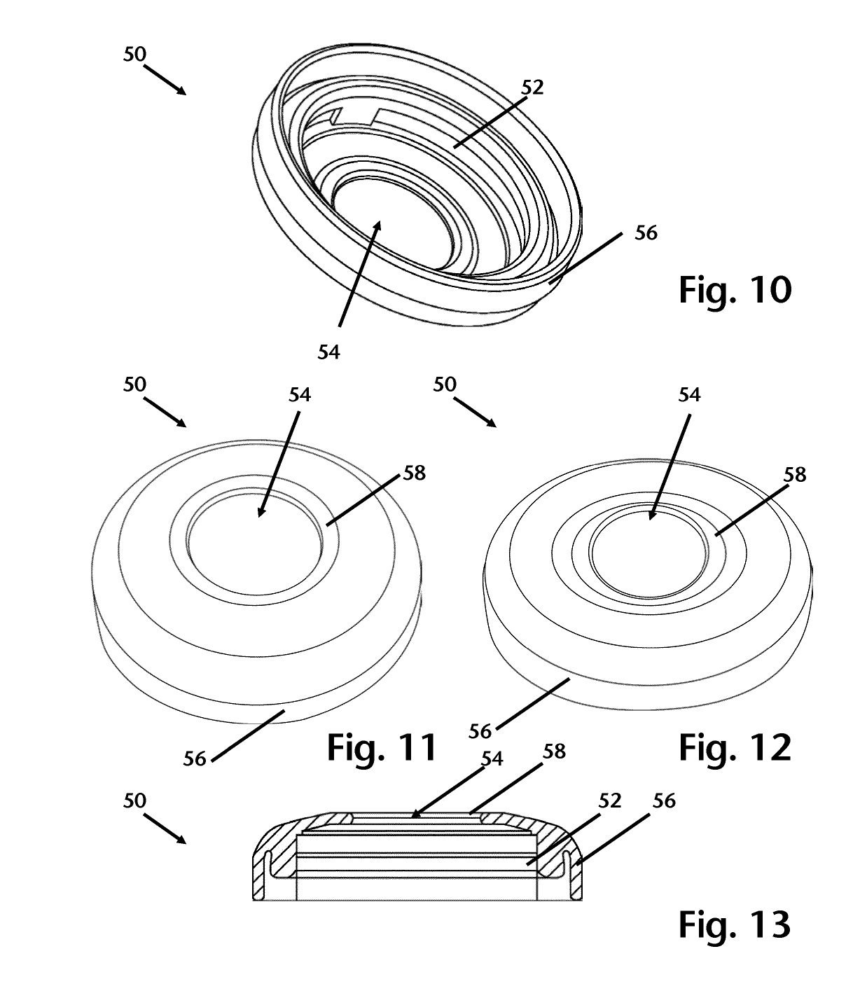

FIG. 9 shows the cover from FIG. 8 in a sectional view;

FIG. 10 shows a shield;

FIG. 11 shows a variation of the shield having a circular design;

FIG. 12 shows a variation of the shield having an oval design;

FIG. 13 shows a sectional view of a shield;

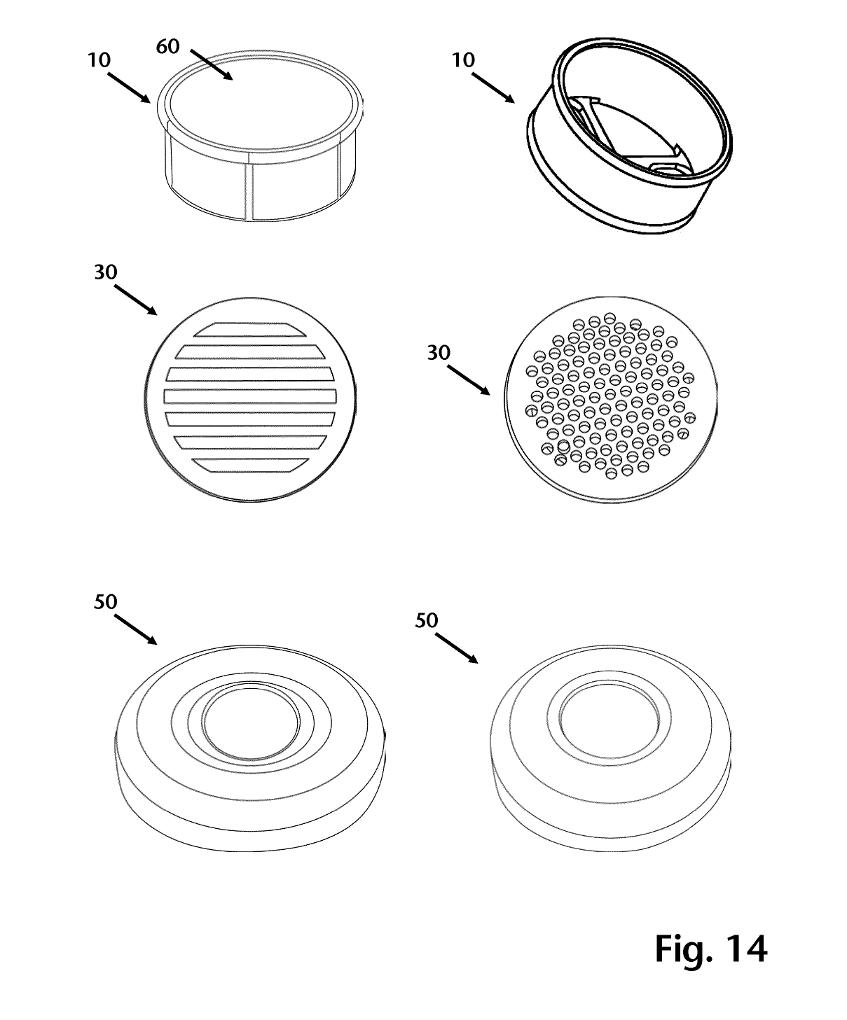

FIG. 14 shows an assembly kit composed of a number of housings, a number of covers, and a number of shields; and

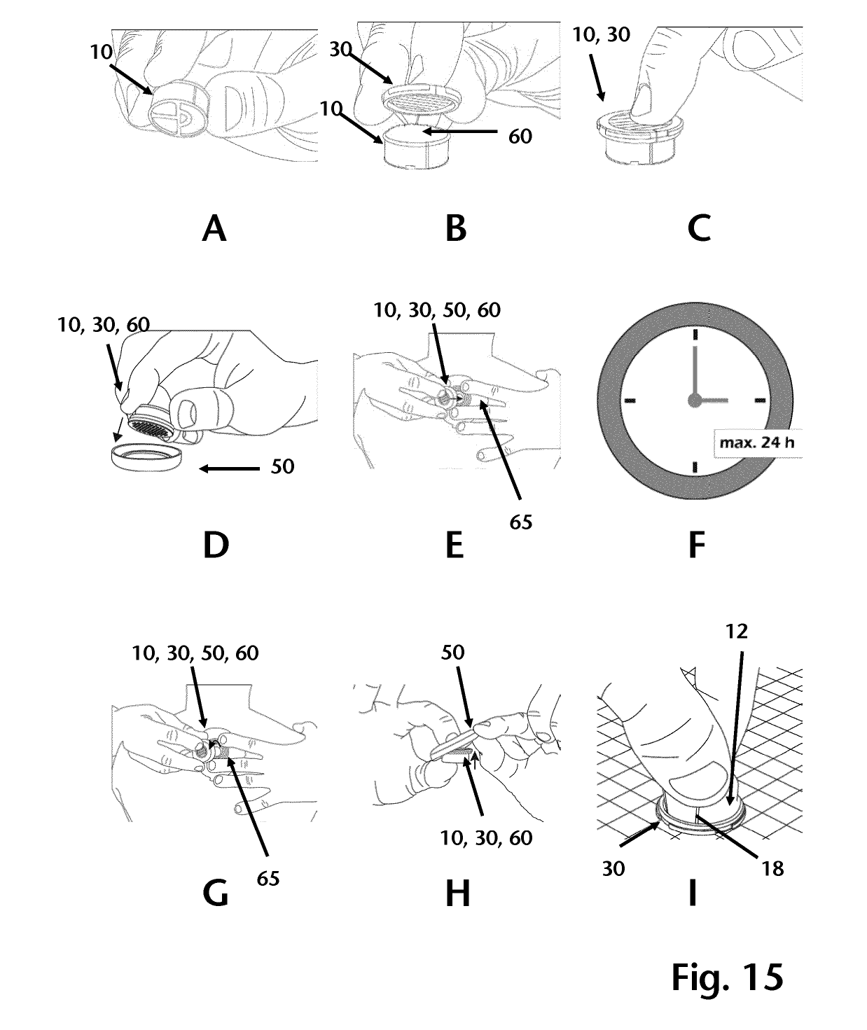

FIG. 15 shows a method for the use of a heat and moisture exchanger comprising the assembly kit from FIG. 14.

FIG. 1 shows a housing 10 of a heat and moisture exchanger for tracheotomy and laryngectomy patients. The housing 10 has a cylindrical housing casing 12 and a filter retaining device 14. A filter, not shown here, in particular made of an open-cell foam, is inserted in the housing 10, and inserted in a tracheostoma device such as a tracheostoma bandage or a tracheal cannula, such that the filter retaining device 14 sits proximal to the tracheostoma. The filter retaining device 14 prevents an unintentional inhalation of the filter. Furthermore, a cover receiver 22 encompassing the outside of the cylindrical housing casing can be seen in FIG. 1, designed as a beading on the distal surface.

FIG. 2 shows the housing 10 seen from above, wherein the filter retaining device 14 can be seen in detail. This has two webs 16 disposed in relation to one another, the web ends 17 of which, facing radially outward, are connected to the inner surface of the cylindrical housing casing 12, or transition into said inner surface. It can be seen that a stop 20 is disposed on one of the webs 16, forming an arc, i.e. a circular ring segment, in this design. Furthermore, it can be seen in FIG. 2 that the cylindrical housing casing 12 has three notches 18, which weaken the cylindrical housing casing 12 in a defined manner. A radial pressure applied to the cylindrical housing casing 12, for example, in the region of the notch 18.1 makes it possible for the cylindrical housing casing 12 to become dented. The defined denting is substantially limited in terms of its extent by the notches 18.2 and 18.3. The notches 18.1, 18.2, and 18.3 face radially toward the stop. Furthermore, the denting is limited by the stop 20, such that an unintended or excessive deformation, or uncontrolled breakage, cannot take place.

FIG. 3 shows a sectional view of the housing in FIG. 2 cut along III-III. This view likewise shows the cylindrical housing casing 12 and the filter retaining device 14 with the web 16. It can further be seen that the housing 10 has a beading on the distal surface, which forms the cover receiver 22. It can furthermore be seen in FIG. 3 that the notches 18.1, 18.2, and 18.3 extend over the entire height of the cylindrical housing casing 12. As can also be seen in FIG. 2, the notches 18.1, 18.2, and 18.3 are formed such that they are continuous from the proximal inner edge 19 to the distal inner edge 21 of the housing 10. FIG. 4 shows a cover for the housing 10 of a heat and moisture exchanger. The cover 30 has a number of holes 34, delimited by lamellas 35. Furthermore, a snap-on element 33 can be seen in FIG. 4, provided in particular for receiving a shield.

FIG. 5 shows the cover 30 from FIG. 4 seen from above. It can be seen that the lamellas 35 run at a diagonal, and the direct view through the holes 34 is relatively narrow, although it can be seen in FIG. 6 that the holes are large enough, but they run diagonally through the cover.

As can furthermore be seen in FIG. 6, the sectional view of FIG. 5 cut along VI-VI, the holes 34 pass through the cover 30 at an angle 36 of approx. 45° to a perpendicular 38 to the surface 40 of the cover 30. Furthermore, the partially encompassing second snap-on element 33 can be seen in FIGS. 4 to 6, running along the outside of the cover rim 31. As can be seen by way of example in FIG. 5, the snap-on element 33 does not entirely encompass the cover rim 31, but instead has interruptions. It can furthermore be seen in FIG. 6 that the cover 30 comprises a cover rim 31 having a circular encompassing first snap-on element 32 on the inside. This snap-on element 32 interacts with the cover receiver 22 of the housing described above.

FIGS. 7 to 9 show another design of the cover, in which the holes 34 are designed as bores. The bores pass through the cover 30 perpendicular to the surface 40 thereof.

FIG. 10 shows a shield 50 that is to be attached to the cover 30. The shield 50 has an at least partially encompassing, inner snap-on receiver 52, which forms a snap-on connection 52 with the second snap-on element 33 of the cover 30. The shield also has a breathing hole 54, which is designed, in particular, such that when it is placed on the cover 30, or after it has been placed on the cover 30, the holes are completely open. In this manner, an unimpaired breathing through the assembled heat and moisture exchanger is enabled. The shield 50 has a rim 56 that encompasses the housing 10 and the cover 30, which ensures in particular, that the housing 10 and the cover 30, and potential build-up of the tracheostoma bandage, the tracheal cannula or the tracheostoma button are not visible. In this manner, the aesthetic desires of a user are satisfied, because the assembled heat and moisture exchanger described herein also fulfills a decorative function.

FIG. 11 shows a variation of the shield 50, wherein the shield 50 is round, in particular approx. circular, in particular in its cross section.

FIG. 12 shows another design of the shield 50, wherein this shield has an oval cross section. In particular, the breathing hole 54 is also oval.

It can furthermore be seen in FIGS. 11 and 12 that the breathing hole 54 has a funnel-shaped design. This funnel shape provides a comfortable seat for the finger on the assembled heat and moisture exchanger, in particular when the user desires to close it.

FIG. 13 shows a sectional view of a shield 50. The breathing hole 54 as well as the snap-on receiver 52 can be seen therein.

FIG. 14 shows an assembly kit for a heat and moisture exchanger. This has a number of housings 10 and a number of covers 30, as well as a number of shields 50. Two variations of the housing 10, the cover 30, and the shield 50 can be seen in the illustrated example. A user, or a medical caregiver, e.g. a physician of the user, can select the appropriate components for the heat and moisture exchanger. The size of the housing or the housing receiver in a tracheostoma bandage or suchlike, as well as the desired functionality as well as the desired design thereof, play a role thereby. It is preferably provided that the housing is designed as a disposable housing. This has a filter 60, supplied separately, or directly therewith, which is inserted in the housing. After use, or after a certain period of use, the housing, together with the filter, or just the filter, is replaced. Furthermore, the user can select a cover according to his own preferences, which on one hand, prevents a direct view of the filter, and on the other hand also prevents the filter from falling out in the distal direction. Furthermore, the cover has a protective function in so far as it prevents large particles, lint or suchlike, and in particular, the finger of the user when closing the heat and moisture exchanger, from coming in direct contact with the filter 60. The user can furthermore select a shield 50, which in particular is intended to satisfy an aesthetic demand. Moreover, the shields also fulfill the technical purpose of making a closing of the heat and moisture exchanger, e.g. with the finger, comfortable. For this, it is provided that, on one hand, the shield 50 is made of a rubbery-elastic material, and on the other hand, the breathing hole 54 has a funnel-shaped design.

FIG. 15 shows the use of the heat and moisture exchanger described herein, or its assembly kit, respectively. Pictogram A shows how a user places the housing 10 on a stable surface. If a filter has not yet been placed in the housing 10, the filter 60 is then inserted, and in the following step, as shown in Pictogram B, the cover 30 is placed on the housing 10, and snapped in place. Pictogram C shows that the snapping in place can take place easily by means of finger pressure. The following Pictogram D shows that the housing 10 provided with the cover 30 is placed in a shield 50. It is also proposed for this that the shield 50 be placed on a stable surface, and the housing 10, together with the cover 30, be pressed into the shield 50. As can be seen in Pictogram D, the cover 30 plays a central role here. This retains both the housing 10 as well as the shield 50. According to a preferred design, it is therefore not possible to connect the housing to the shield 50 without the cover 30. The cover 30 thus provides a coupling function.

Pictogram E shows that the fully assembled heat and moisture exchanger 10, 30, and 50 is placed in a tracheostoma bandage 65. Pictogram F illustrates that the heat and moisture exchanger can be worn for a certain period of time, wherein a maximum period of 24 hours is recommended. Depending on the soiling or formation of secretions, an earlier or later replacement can take place. After this time has elapsed, as is illustrated in Pictogram G, the heat and moisture exchanger is either removed, or the shield 50 is released from the cover 30. At this point, the housing 10, in particular the cylindrical housing casing 12, is dented in the region of the notches 18, until the cover is released from the housing 10.

1. A housing (10) of a heat and moisture exchanger for tracheotomy and laryngectomy patients, comprising a cylindrical housing casing (12) and a filter retaining device (14), wherein the filter retaining device (14) comprises at least one web (16), allocated to a base surface of the housing (10), and wherein the cylindrical housing casing (12) has at least three notches (18), which enable a defined denting of the cylindrical housing casing (12).

2. The housing (10) according to claim 1, characterized in that the notches (18) are disposed at an angle of approx. 15° to approx. 75° to one another.

3. The housing (10) according to one or more of the preceding claims, characterized in that the notches (18) are disposed on the cylindrical housing casing (12), distributed over an angle of approx. 90° to approx. 180°.

4. The housing (10) according to one or more of the preceding claims, characterized in that at least one notch (18) is disposed on the inside of the cylindrical housing casing (12).

5. The housing (10) according to one or more of the preceding claims, characterized in that at least one notch (18) is disposed on the outside of the cylindrical housing casing (12).

6. The housing (10) according to one or more of the preceding claims, characterized in that the filter retaining device (14) comprises a stop (20) for the denting of the cylindrical housing casing (12).

7. The housing (10) according to claim 6, characterized in that the stop (20) is disposed on the web (16).

8. The housing (10) according to one or more of the preceding claims, characterized in that the housing is made of a material selected from a group comprising at least polypropylene and/or polyvinyl chloride.

9. The housing (10) according to one or more of the preceding claims, characterized in that the filter retaining device (14) has a number of webs (16).

10. The housing (10) according to claim 9, characterized in that web ends (17) of the webs (16) are distributed over an angle of approx. 120° to approx. 200° on an inner circumference of the cylindrical housing casing (12).

11. The housing (10) according to one or more of the preceding claims, characterized in that the housing has a cover receiver (22) for a cover (30).

12. The housing (10) according to claim 11, with a cover (30), wherein the cover (30) is snapped in place on the housing (10).

13. A method for releasing a cover (30) from a housing (10) according to one of the claims 1 to 11, characterized in that a force is exerted, perpendicular to an outer surface of a cylindrical housing casing (12), such that the cylindrical housing casing (12) is dented in a manner defined by the notches (18), and the cover (30) is released from a snap-on connection (22, 32).
